(19) United States
(12) Patent Application Publication
(10) Pub. No.: US 2020/0040292 A1
(43) Pub. Date: Feb. 6, 2020

(54) MICROBIOREACTOR MODULE

(71) Applicant: Yoen Ok ROTH, Dusseldorf (DE)

(72) Inventor: Yoen Ok ROTH, Dusseldorf (DE)

(21) Appl. No.: 16/340,206

(22) PCT Filed: Oct. 6, 2017
(86) PCT No.: PCT/EP2017/075442

§ 371 Date: Apr. 8, 2019

(30) Foreign Application Priority Data
Oct. 12, 2016 (DE) 10 2016 119 391.7

(51) Int. Cl. C12M 1/00; C12M 1/12; C12M 1/34

(52) U.S. Cl. CPC C12M 23/14; C12M 23/38; C12M 25/14; C12M 41/00; C12M 29/04; C12M 29/12; C12M 27/22

(57) ABSTRACT

The invention relates to a microbioreactor module for the three-dimensional cultivation of cells, especially stem cells. Said microbioreactor is intended for single use and, in an embodiment of the invention, can be used as a multi-microbioreactor.

The use of stem cells is becoming increasingly important in medical research, especially in the field of regenerative medicine. Also in pharmaceutical research and in the cosmetics industry the cultivation of stem cells is used in some areas to carry out, for example, ADME/Tox studies, i.e. to test new potential active substances for their properties with regard to absorption, distribution, metabolization, excretion and toxicity.

Embryonic and so-called induced pluripotent stem cells are characterized by their almost infinite potential for self-renewal, proliferation and differentiation, also in cell culture. Tailor-made methods and devices for cultivation, however, play a decisive role in reliably directing differentiation towards a certain tissue type and at the same time preventing undesired malignant tumor formation (cf. Lutolf, M. P.; Gilbert, P. M.; Blau, H. M., Designing materials to direct stem-cell fate. Nature 2009, 462 (7272), 433-441).

Especially for use in regenerative medicine, there is a high interest in cellular material obtained in vitro, which exhibits a high degree of consistent quality, so that there is a great demand for standardized cell culture methods that guarantee long-term cultivation and long-term differentiation of stem cells under controlled conditions and ideally enable process automation or at least process parallelization.

Therefore, solutions for the targeted cultivation of different cell types from pluripotent stem cells are intensively sought. First, partly very complex possibilities and devices have been developed for tissue production. In particular, so-called organ chips are used which enable the cultivation of, for example, lung, liver, heart, skin or bronchial tissue (cf. Lang, Q.; Ren, Y.; Wu, Y.; Guo, Y.; Zhao, X.; Tao, Y.; Liu, J.; Zhao, H.; Lei, L.; Jiang, H., A multifunctional resealable perfusion chip for cell culture and tissue engineering. RSC Advances 2016, 6 (32), 27183-27190).

In vivo, stem cells can be found in tissue-specific stem cell niches of the body. In such a niche, the stem cells are not only physically bound, but the special microenvironment of the niche determines the development of the stem cells through its regulatory network of biochemical processes and signals induced by chemokines, cytokines, growth factors, transmembrane receptors, as well as the extracellular matrix. Therefore, for the cultivation of stem cells in vitro, attempts are being made to artificially imitate the microenvironment of the stem cell niches (cf. Lutolf, M. P.; Gilbert, P. M.; Blau, H. M., Designing materials to direct stem-cell fate. Nature 2009, 462 (7272), 433-441).

When imitating the natural environment of stem cell niches, other cell types as well as gradients and concentration gradients in the medium play an overriding role with regard to the effectiveness of the process in addition to cellular microenvironment.

EP 2 181 188 B1 discloses a microbioreactor which is arranged as a microfluidic system and is suitable for the cultivation of advanced cell cultures, especially 3D cell cultures and stem cell cultures. A special feature is the construction of a media circuit for perfusion of the microbioreactor. The sample carrier on which the cell growth takes place is one or more 3D cell chips stacked one upon the other. The arrangement of several mutually independent microbioreactors on a microtiter plate makes it possible to use a multimicrobioreactor, especially for high-throughput screening.

US 2011/0136226 A1 discloses an artificial stem cell niche which comprises a rotating culture chamber in which a scaffold with mesenchymal connective tissue stem cells is attached on which umbilical cord blood stem cells are cultivated. The culture chamber is supplied via a fluid supply system in which the nutrient supply and gas and waste exchange takes place through a dialysis membrane and a second fluid system enables cell harvesting from the suspension inside the culture chamber.

US 2011/0207166 A1 discloses an artificial microenvironment which corresponds to a replica of a niche in the bone marrow microenvironment consisting of a scaffold coated with mesenchymal stem cells and a culture medium which enables proliferation of the stem cells into the culture. The artificial niche is suitable for the cultivation of hematopoietic and leukemic cells. The scaffold consists of a net-like, expandable matrix made of an elastomeric material, e.g. polycarbonate or polyurethane.

However, the aforementioned well-known bioreactors all have the disadvantage that their structure is very complex and therefore an easy handling, production and, in particular, use as a disposable system is limited.

DE 10 2014 001 615.3 discloses a device for the cultivation of adherent cells which is operated as a disposable system in a continuous process. A special feature of this device is the homogenization of the culture medium in the reactor vessel by means of a horizontal and a vertical, cushion-shaped pump element, each being operated by compressed air. Corresponding flow distributors ensure uniform mixing. The gassing of the culture medium takes place through semi-permeable membrane hoses. However, this device has the disadvantage that it is only designed for the cultivation of adherent cells, but not of stem cells with their special requirements.

U.S. Pat. No. 4,649,117A discloses a reactor which can be used as a fermenter for the cultivation of cells, wherein a particularly shear force-free mixing is achieved by the fact that the reactor consists of an inner and an outer chamber and a gentle gas stream is introduced centrally from below. However, this reactor is not suitable for the cultivation of adherent cells or stem cells, as no corresponding growth areas are provided.

It is the object of the present invention to use the advantages of the known devices, to avoid the disadvantages and to provide a simpler, flexibly useable solution for the problem of stem cell culture. The microbioreactor module according to the invention can be used advantageously as a disposable system. A parallel arrangement of several modules in a common or in separate culture rooms allows the use as a multimicrobioreactor. In an arrangement as a multimicrobioreactor, the microbioreactor module according to the invention is particularly suitable for screening and selecting optimal cultivation conditions due to its defined and controllable microenvironment.

### EMBODIMENTS

For a better understanding of the present invention, this invention will be explained in more detail using the embodiments shown in the following figures. Identical parts are provided with identical reference signs and identical component designations. Furthermore, some features or combinations of features from the different embodiments shown and described may represent independent solutions, inventive solutions or solutions according to the invention.

The associated drawings show in

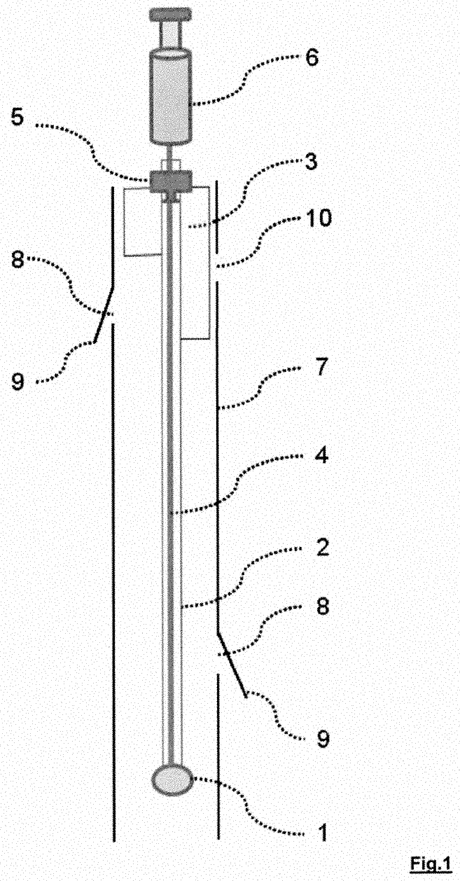

FIG. 1 a schematic representation of the microbioreactor module, wherein the cultivation of the stem cells takes place in a cultivation container (1).

FIG. 2 a schematic representation of a multimicrobioreactor, in which several microbioreactor modules are introduced into a cultivation room according to DE 10 2014 001 615.3.

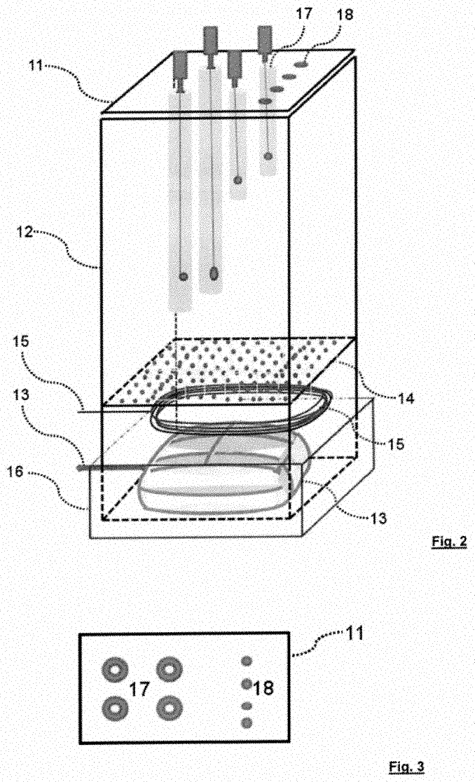

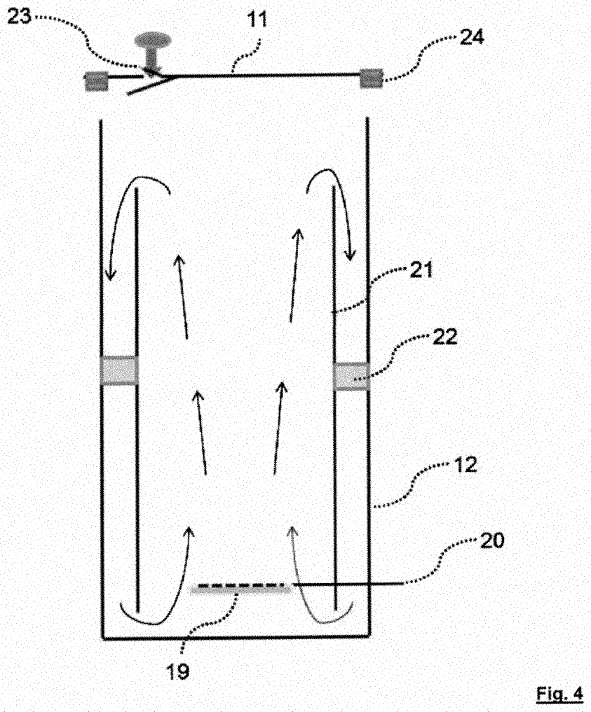

FIG. 3 a schematic representation of a plan view of the cover (11), in which, in addition to an upwardly open number of module slots (17), there are also various connection options for probes (18), which enable online process control at various positions in the reactor vessel (12).

FIG. 4 a schematic representation of a microbioreactor module, wherein the mixing of the medium in the reactor vessel (12) is ensured by an arrangement corresponding to a bubble column or a loop reactor.

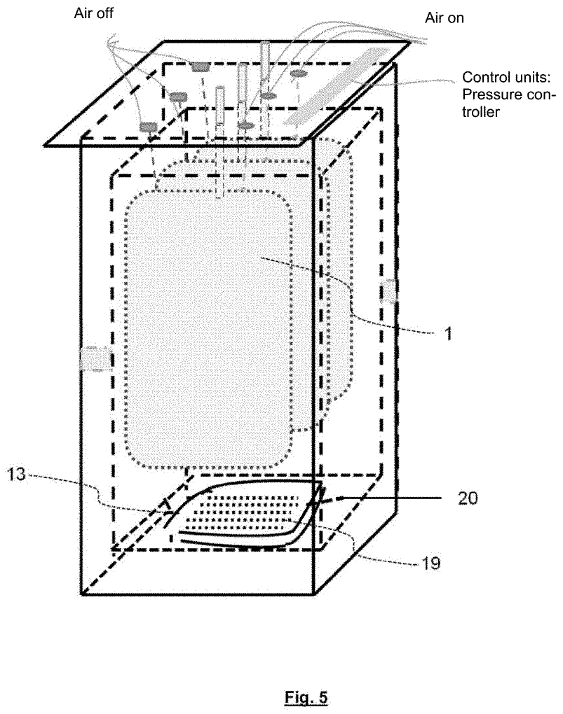

FIG. 5 a schematic representation of a scale-up bioreactor containing the modules according to the invention.

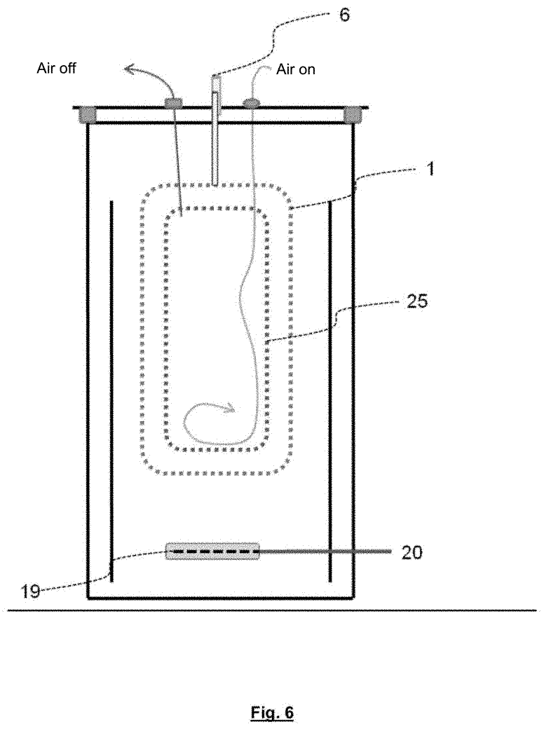

FIG. 6 a schematic representation of a side view of a scale-up bioreactor, wherein a gas-permeable air bag is located in the interior of the cultivation container.

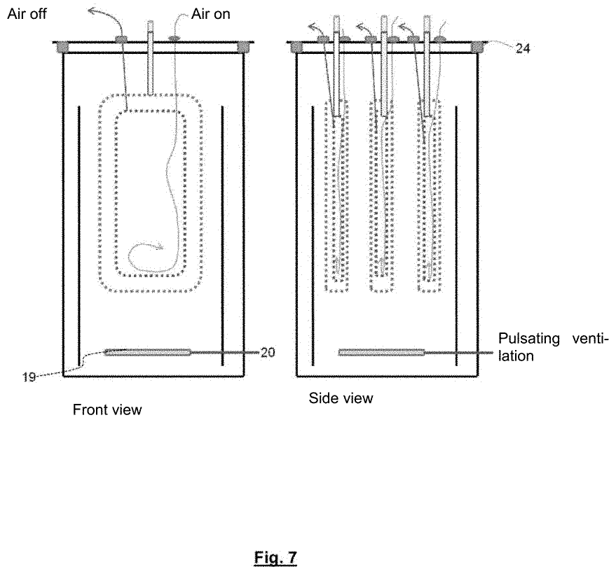

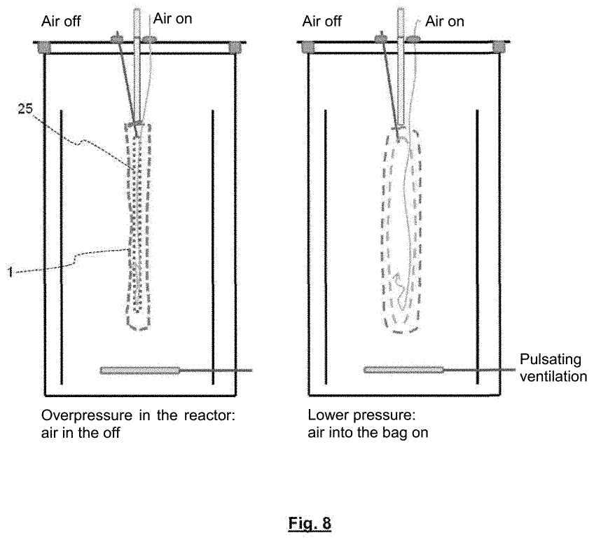

FIG. 7 a schematic representation of the front view and the side view of a scale-up bioreactor.

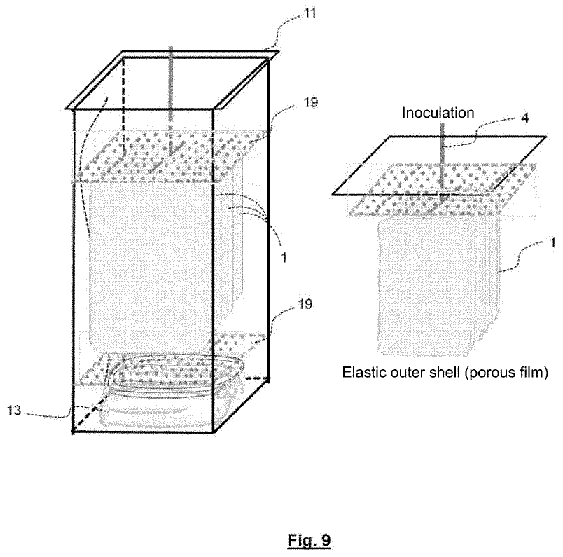

FIG. 8 a schematic representation of a scale-up bioreactor, wherein the left illustration shows the state without active air supply (overpressure in the reactor) and the right illustration shows the state with active air supply, which can be used inter alia to imitate the blood pressure (systole and diastole).

FIG. 9 shows a schematic representation of a scale-up cultivation unit with a large growth surface.

FIG. 1 shows a microbioreactor module, wherein stem cell cultivation takes place in a cultivation container (1) imitating the microenvironment of a stem cell niche. The cultivation container (1) of the microbioreactor module consists of a bag of a semi-permeable natural or synthetic membrane material, so that an exchange of small molecules, such as amino acids or glucose, with the environment is possible, but the cell culture is physically fixed. The cultivation container (1) is equipped with a biocompatible carrier material or scaffold (preferably organic or inorganic polymer materials) and can also be used for co-cultivation with mesenchymal stem cells, for example, by appropriately colonized carriers.

The cultivation container (1) is attached to a gas-permeable mounting tube (2), preferably made of plastic, which ends at the other end in a plug (3) made of plastic, rubber or silicone. The plug (3) is asymmetrical in thickness. In a preferred embodiment, the gas-permeable mounting tube (2) is made of an elastic or semi-elastic material.

Inside the mounting tube (2) there is a discharge and supply line (4) which allows the inoculation of cells, the supply of bioactive molecules and nutrients, and the taking of samples. The discharge and supply line (4) is made of an elastic or semi-elastic material in a preferred embodiment.

The line (4) ends on the upper side of the plug (3) in a connecting piece (5) with a thread, which is used as an adapter for mounting, for example, a syringe (6), by means of which the supply and discharge of substances, medium and cells is controlled. The opening of the line (4) in the connecting piece (5) is provided with an elastic closure material which allows piercing with the mounted syringe (6) and closes when the syringe (6) is removed.

The core piece is inserted with the plug into a sheathing (7) which is opened downwards. The sheathing (7) has a total of three openings on the side walls. Two of the openings are located at the level of the plug (3), another one in the lower third of the sheathing (7). The through-flow openings (8) are provided with a flap (9) which opens or closes depending on the direction of flow of the external medium. The third opening does not have such a flap and serves as a discharge (10) for resulting wastes, e.g. unwanted metabolites. The asymmetrical thickness of the plug (3) and its condition allow a targeted opening or closing of either the through-flow opening (8) or the waste discharge (10) by rotating the plug (3) in the sheathing (7). The sheathing (7) preferably consists of a semi-elastic plastic material.

The cultivated cells can be harvested either by partial removal via the discharge and supply line (4) for continuous process control, or by separating the cultivation container (1) from the mounting tube (2).

The microbioreactor unit according to the invention can be used in a variety of cultivation systems. The unit is inserted parallel to the respective main flow direction of the device to ensure that the through-flow openings (8) function in accordance with the invention.

FIG. 2 shows a multimicrobioreactor in which several microbioreactor modules are placed in a cultivation room according to DE 10 2014 001 615.3. In this preferred embodiment, one or more different microbioreactor modules according to the invention are fixed in parallel in a plastic cover (11) in openings provided with seals and are inserted into a replaceable reactor vessel (12). In the reactor vessel (12) filled with a medium, uniform, gentle and shear force-free homogenization is achieved by means of a compressed air-driven pump element (13) located at the bottom. The flow direction of the homogenization, which is intensified by perforated plates acting as flow distributors (14), runs parallel to the orientation of the microbioreactor modules. Semipermeable membrane hoses (15) enable bubble-free gassing of the medium in the reactor vessel. Temperature control can be achieved by introducing the reactor vessel (12) into a heating element (16), wherein the heating element (16) preferably only covers the lower end of the reactor vessel (12), so that a minimum vertical temperature gradient occurs inside the reactor vessel (12).

The growth conditions in the individual microbioreactor modules can be individually adapted. The composition of nutrients in the individual cultivation containers (1), which may be equipped with different carrier materials and cell cultures, can be adapted by separate discharge and supply lines (4) of the individual modules. The length of the microbioreactor modules can vary and thus a different immersion depth into the medium in the reactor vessel (12) can be achieved. Thus, the growth conditions with respect to temperature (corresponding to the vertical temperature gradient) and pressure (corresponding to the hydrostatic pressure) can be measured with the probe and individualized.

FIG. 3 shows a top view of the cover (11), in which, apart from an upwardly open number of module slots (17), various connection options for probes (18) are also provided, enabling online process control at various positions in the reactor vessel (12).

The number of individual microbioreactor modules in a single reactor vessel (12) is limited only by the size of the reactor vessel, wherein the volume of a single module can also vary from the microliter to milliliter scale.

FIG. 4 shows another possible design of a reactor vessel for the application of the microbioreactor module according to the invention, wherein the mixing of the medium in the reactor vessel (12) is ensured by an arrangement corresponding to a bubble column or a loop reactor. An upwardly opened plate with small openings arranged like a grid serves as a bubble generator (19) through which compressed air flows pulsatingly via a compressed air supply (20). The cover (11) is connected to the reactor vessel (12) at the seals (24) and corresponds to the arrangement in FIGS. 1-3, but additionally contains a pressure relief valve (23). The bubbles produced by the bubble generator (19) cause a mixing flow in the vertical direction, which flows upwards within an inner reactor shell (21), which is open at the top and bottom and fixed to the reactor vessel by mounting brackets (22). In the space between the inner reactor shell (21) and the reactor vessel (12), a corresponding counterflow occurs. Excess air is passed to the outside through the pressure relief valve (23). The opening and closing of the pressure relief valve (23) is accompanied by the pulsating supply of compressed air, so that the pressure increased during the supply of air is replaced by a phase of relaxation. One or more microbioreactor modules are introduced in the cover (11) parallel to the direction of flow, by analogy with the preferred embodiment shown in FIGS. 1-3.

In a preferred embodiment, the microbioreactor module serves to screen a suitable microenvironment for the respective cell type to be cultivated. The composition and concentration of different growth factors, the presence of extracellular matrix factors, dissolved oxygen concentration, pH value, osmolarity and the continuous supply of nutrients as well as the removal of metabolites are optimized.

Another preferred embodiment is the scale-up version of the bioreactor, which allows the cultivation of the desired cell type under optimized conditions on a large scale. FIGS. 5-9 show schematic diagrams of a scale-up bioreactor. The scale-up bioreactor contains one or more modules according to the invention that contain a larger volume, thus enabling a high cell yield under optimized, controlled, and reproducible conditions. The three-dimensional culture in a microbioreactor allows a fast conversion into production and a simple scale-up process. Furthermore, the controllable parameters during cultivation allow easy and gentle cell harvesting.

In another embodiment, the scale-up bioreactor enables continuous fermentation due to the controllable cultivation conditions. In one embodiment, the scale-up bioreactor has a bubble generator (19) above and below the cultivation containers (1).

Another advantage of the microbioreactor module in general, and of the scale-up bioreactor in particular, is the possibility of varying the pressure in the cultivation container to thereby simulate, for example, the blood pressure that varies in the body of an individual with systole and diastole (blood pressure 120/60 mmHg, i.e. 1160/60 mbar). In the microbioreactor module this is made possible by the compressed air supply (20) and the bubble generator (19). In the scale-up bioreactor there is a gas-permeable air bag (25) in the cultivation container, which by changing the volume and pressure in the air bag simulates the pulsating physical property of the pulsating blood in the cultivation container. This is accompanied by homogenization within the cultivation container. Furthermore, the material exchange as well as the oxygen exchange is promoted by the generated pressure gradient on the surface of the cultivation container. The air bag also has the advantage that it enables the gas exchange of carbon dioxide (CO₂) and ammonium (NH₄).

### LIST OF REFERENCE NUMERALS

1: Cultivation container
2: Mounting tube
3: Plug
4: Discharge and supply line
5: Connecting piece
6: Syringe
7: Sheathing
8: Through-flow opening
9: Flap
10: Discharge
11: Cover
12: Reactor vessel
13: Pump element
14: Flow distributor
15: Membrane hose
16: Heating element
17: Module slots
18: Probes
19: Bubble generator
20: Compressed air supply
21: Inner reactor shell
22: Mounting bracket
23: Pressure relief valve
24: Seal
25: Gas-permeable air bag

1. A microbioreactor module comprising a cultivation container, a mounting tube, an asymmetrical plug, and a sheathing, the microbioreactor module being characterized in that
the cultivation container has an outer shell of a semi-permeable membrane material enclosing a biocompatible carrier material or scaffold,
the mounting tube is attached to the cultivation container and encloses a discharge and supply line which allows samples to be taken and cells or bioactive molecules and nutrients to be supplied to the cultivation container,
the asymmetrical plug has an asymmetrical length, projects into the interior of the microbioreactor module, fixes the mounting tube in an opening, and seals the microbioreactor module to the outside by contact with the sheathing, and opens or closes openings in the wall of the sheathing depending on the positioning.

2. The microbioreactor module according to claim 1, characterized in that the outer shell of the cultivation container comprises a bag of a semi-permeable organic or inorganic membrane material with an exclusion size of 1-50 kDa, for example a dialysis hose material.

3. The microbioreactor module according to claim 1, characterized in that the carrier material in the cultivation container is comprised of natural or synthetic polymers or inorganic materials or a combination thereof, or a corresponding scaffold.

4. The microbioreactor module according to claim 1, characterized in that the mounting tube, the discharge and supply line and the sheathing comprises an elastic, gas-permeable material, and that the sheathing comprises a plurality of through-flow openings (8) which can be closed by flaps.

5. The microbioreactor module according to claim 1, characterized in that the carrier material or the scaffold is coated with stem cells.

6. The microbioreactor module according to claim 1, characterized in that the cultivation container contains a gas-permeable air bag.

7. A microbioreactor comprising one or more microbioreactor modules according to claim 1, which are arranged in parallel and introduced into a common cultivation room.

8. The microbioreactor according to claim 7, characterized in that one or more microbioreactor modules are fixed in a common plastic cover, in which connection possibilities for measuring probes.

9. The microbioreactor according to claim 7, characterized in that the common cultivation room is a reactor vessel filled with medium, in which homogenization is ensured by a pump element operated by compressed air in combination with a flow distributor.

10. The microbioreactor according claim 7, characterized in that the common cultivation room is a reactor vessel filled with medium, in which homogenization is achieved by a bubble generator by passing pulsating compressed air through a plate opened upwards by way of holes arranged in a grid-like manner.

11. The microbioreactor according claim 7, characterized in that an upwardly and downwardly opened inner reactor shell is fixed in the interior of the reactor vessel by means of mounting brackets and a flow circulation in the medium is thereby achieved.

12. The microbioreactor module according to claim 3, characterized in that the carrier material comprises collagen, elastin, fibrin, alginate, silk, glycoaminoglucan, hyaluronic acid, chitosan, cellulose, fucoidan or silaffin.

13. The microbioreactor module according to claim 4, wherein the elastic, gas-permeable material comprises silicone.

14. The microbioreactor module according to claim 5, wherein the stem cells are mesenchymal stem cells.

15. The microbioreactor module according to claim 8, further comprising pressure relief valves.
